(19) United States
(12) Patent Application Publication
(10) Pub. No.: US 2022/0054698 A1
(43) Pub. Date: Feb. 24, 2022

(54) CATALYTIC HYDROGEN PEROXIDE GENERATION FOR DISINFECTION

(71) Applicant: CASPR Group Inc., Dallas, TX (US)

(72) Inventor: Christophe Suchy, Irving (US)

(21) Appl. No.: 17/407,345

(22) Filed: Aug. 20, 2021

Related U.S. Application Data
(60) Provisional application No. 63/068,311, filed on Aug. 20, 2020.

(51) Int. Cl. A61L 9/20; B01J 21/06; B01J 35/00; B01J 35/04; A61L 2/20; A61L 2/26; A61L 2/08; A61L 9/015

(52) U.S. Cl. CPC A61L 9/205; B01J 21/063; B01J 35/004; B01J 35/04; A61L 2/208; A61L 2202/11; A61L 2/088; A61L 9/015; A61L 2209/211; A61L 2209/12; A61L 2209/16; A61L 2/26

(57) ABSTRACT

In various implementations, systems and processes may generate hydrogen peroxide using a catalyst that includes titanium dioxide, silver, antimony, copper, and/or rhodium. The systems and processes may utilize an air stream in the presence of UV light and a catalyst to generate hydrogen peroxide. The generated hydrogen peroxide may be utilized to disinfect air and surfaces.

### CROSS-REFERENCE TO RELATED APPLICATION

This application is claims the benefit of priority to U.S. Provisional Patent Application No. 63/068,311 entitled “Catalytic Hydrogen Peroxide Generation for Disinfection” and filed on Aug. 20, 2020, which is incorporated fully herein.

### TECHNICAL FIELD

The present invention relates to catalytic generation of hydrogen peroxide and/or disinfection of air and surfaces.

### BACKGROUND

Hydrogen peroxide is a known disinfectant for surfaces and air. However, current air disinfection systems produce negligible amounts of hydrogen peroxide and thus ineffective disinfection and/or excessive amounts of ozone.

Pathogens, including bacteria, viruses, and fungi whether airborne, waterborne, or present on fomites or surfaces are a risk to healthcare personnel and patients, workers and bystanders, passengers, and, in general, occupants in public and private spaces. The reduction of the spread of transmitted diseases and implementing effective means to control infection pathways and patterns becomes a necessity, especially in times of pandemic risks. Some volatile organic compounds (VOCs) are pollutants and dangerous to human health and/or cause harm to the environment. Filtering and cleaning ambient air, and air management or heating, ventilation, and air conditioning (HVAC) systems providing air exchange rates are engineering methods to reduce pathogens and pollutants. In the 1990s, researchers at the Wisconsin Center for Space Automation and Robotics, a NASA research partnership center at the University of Wisconsin in Madison, developed an air purifier device that eliminates pathogens in the ambient air of spacecrafts. The device draws air through thin tubes of titanium dioxide, TiO₂ that are exposed to ultraviolet light, performing a photo-catalytic oxidation (PCO) process that decomposes ethylene, C₂H₄ the decaying gas produced by plants in greenhouse experiments in space. The first device was used aboard Space Shuttle mission STS-73 in 1995, and since then in numerous International Space Station (ISS) expeditions. Hydrogen peroxide, H₂O₂ has a highly efficacious profile in disinfection and falls at the top range of disinfectants. Aqueous solutions of hydrogen peroxide, H₂O₂, even at low-level concentrations, are reported to kill pathogenic microorganisms reliably in short time, as the reduction/oxidation potential (ROP) of hydrogen peroxide, H₂O₂ has an ROP of 1.77V on the scale between zero for diatomic molecular hydrogen, H₂ and the strongest oxidizer, fluorine, F at 2.87V. Foggers producing a mist or vapor of disinfectants, such as aqueous solutions of hydrogen peroxide, H₂O₂, are available to disinfect surfaces and rooms after contamination. Paul J. Crutzen, one of the Nobel Prize winners in Chemistry 1995 described the oxidizer cycling, namely the hydroxyl radical, .HO as the “detergent of the atmosphere”, cleansing it from the greenhouse gases carbon monoxide, CO and other carbon-based molecules, such as methane, CH₄ by decomposing, i.e. oxidizing its chemical structure. These oxidizers are formed in the atmosphere when ultraviolet light (UV) from the sun strikes ozone, O₃ in the presence of water vapor, H₂O. As radicals are highly reactive, having unpaired electrons which tend to transfer to other molecules, they have a lifespan of only a few seconds. However, due to complex photochemical processes, they form other oxidizers, e.g. the much more stable hydrogen peroxide, H₂O₂ in its gaseous form. To understand the air cleaning mechanism resulting from humidity (e.g., gaseous water, H₂O and diatomic molecular oxygen, O₂) in the sunny daylight, meteorologists have measured the concentrations of gaseous hydrogen peroxide, H₂O₂ in the atmosphere and found, for example, that gas-phase hydrogen peroxide, H₂O₂ concentrations range from less than 0.05 to approximately 1.0 ppbv (parts per billion by volume), and respectively from less than 0.05 to 2.0 ppbv at the urban and rural sites in North Carolina, observing, in general, a clear diurnal and seasonal trend. Although these ranges represent a very low concentration trace of gaseous hydrogen peroxide, H₂O₂ in air, a concentration of 2 ppbv at sea level pressure is equivalent to approximately 54 billion (10⁹) hydrogen peroxide, H₂O₂ molecules per cubic centimeter, cm³. Although the United States Occupational Safety and Health Administration (OSHA) has set a permissible exposure limit (PEL) for workplaces at 1 ppmv (parts per million by volume) for hydrogen peroxide, H₂O₂ in vapor, the Risk Assessment and Science Support Branch (RASSB) of the United States Environmental Protection Agency (EPA) assesses the inhalation risk-based level of concern at 7 ppbv, i.e. 0.7% of the OSHA PEL, based on a 2006 toxicological review.

### SUMMARY

In various implementations, systems and processes may generate hydrogen peroxide using a catalyst that includes titanium dioxide, silver, antimony, copper, and/or rhodium. The systems and processes may utilize an air stream in the presence of light and a catalyst to generate hydrogen peroxide. In some implementations, the generated hydrogen peroxide may be utilized to disinfect air and surfaces.

In various implementations, the system may include a lamp and a catalyst that may be coated on a substrate. The lamp may include a UV lamp, such as a mercury arc UV lamp. The sleeve (e.g., tube) of the lamp that houses the UV lamp or portions thereof (e.g., mercury, filament, etc.) may include quartz and fused silica. The substrate may include openings and/or channels to allow air to flow through the substrate. Air may interact with UV light emitted from the lamp and the catalyst on the substrate to generate hydrogen peroxide. The hydrogen peroxide and/or other components of the resulting air stream may pass through the substrate to be emitted to allow disinfection of surfaces and/or the air.

One or more implementations of the system and methods may include one or more of the following features. The catalyst may include titanium dioxide doped with silver, antimony, copper, and/or rhodium. In some implementations, the catalyst of titanium dioxide may be doped with silver, antimony, copper, and rhodium. The catalyst may be coated on at least a portion of an exterior surface of the substrate. The substrate may be similar to a honeycomb in shape. The sleeve may include less (e.g., by length) exposed fused silica than quartz. For example, a portion of the sleeve may be silica and a second portion may be quartz.

In various implementations, a disinfecting system may include one or more lamps, one or more sleeves, and one or more substrates. The disinfecting system may produce hydrogen peroxide for disinfecting the air and/or surfaces contacting the produced hydrogen peroxide. The lamps of the disinfecting system may include one or more UV lamps. The UV lamp selected may emit at least a first band of UV light and a second band of UV light. In some implementations, the first band of UV light comprises approximately 253.7 nm and the second band of UV light comprises approximately 185 nm. The sleeve(s) may include a first zone and a second zone. The first zone may allow the first band of UV light to pass through the first zone and inhibits the second band of UV light from passing through the first zone. The second zone may allow the second band of UV light and the first band of UV light to pass through the second zone. At least a portion of the light from the lamps may be emitted to at least a portion of the one or more sleeves such that at least a portion of the light emitted will be allowed to pass through the first zone and/or the second zone. Light passing through the sleeve may be allowed to shine on at least a portion of a substrate. The substrate may include one or more channels and a catalytic surface. The catalytic surface may be disposed on at least a portion of the substrate. The catalytic surface may include a doped titanium dioxide catalyst. The doped titanium catalyst, in some implementations, may include titanium dioxide doped with approximately 1 mol % to approximately 25 mol % silver, approximately 1 mol % to approximately 25 mol % rhodium, approximately 0.1 mol % to approximately 2 mol % copper, and approximately 1 mol % to approximately 25 mol % antimony. Air may pass through one or more of the channels of the substrate (e.g., to be disinfected and/or to carry hydrogen peroxide to a surface to be disinfected). Hydrogen peroxide may be generated by contacting the air with the catalytic surface and the UV light proximate the substrate.

Implementations may include one or more of the following features. The lamp may include a mercury arc lamp. The mercury lamp may include a low-pressure mercury vapor lamp. The sleeve(s) of the disinfecting system may be include a sleeve in which zones are spliced together and/or may include a set of sleeves (e.g., in which one or more sleeves are disposed inside one or more other sleeves). The first zone may include quartz. In some implementations, the first zone may consist of quartz. In some implementations, the amount of quartz in the first zone may be greater than other components of the first zone. The second zone may include fused silica. In some implementations, the second zone may consist of fused silica. In some implementations, the amount of fused silica in the second zone may be greater that other components of the second zone. In some implementations, the sleeves may be coupled and/or arranged such that a least a portion of the light emitted by the lamp(s) will pass through the second zone and not pass through the first zone (e.g., an exposed second zone may exist). Allowing this part of the emitted light to pass through the second zone without also passing through the first zone may result in light that passes through the sleeves collectively to include a fixed ratio of approximately 253.7 nm UV light to the approximately 185 nm UV light. The length of the exposed second zone may control this ratio. For example, when a sleeve is a dual zone sleeve, the sleeve may include a first zone spliced or otherwise coupled to the second zone. The length of the second zone may be identified as the exposed second zone. As another nonlimiting example, the first zone may be disposed on a first sleeve and the second zone may be disposed on a second sleeve that is partially disposed in the first sleeve. The portion of the second sleeve that is not disposed within the first sleeve may be identified as the exposed second zone. The exposed second zone may have a length of approximately 1% to approximately 5% of the length of the sleeves of the lamp (e.g., the total length of a fused lamp, the overall length of a set of sleeves in which at least one sleeve is disposed in another sleeve). In some implementations, a length of the exposed second zone may be approximately 2.5% of a length of the sleeves. The length of the inner sleeve that includes the second zone that is not disposed in the outer sleeve may be less than the length of the outer sleeve that includes the first zone, when the sleeves are disposed in an at least partially nesting manner. In some implementations, the first zone and/or the second zone may inhibit other bands of light (e.g., by absorbing and/or not allowing bands of light other than 253.7 nm and/or the second band of UV light comprises approximately 185 nm to pass). The substrate may be grid shaped (e.g., the cross-section of at least a portion may be grid shaped). For example, the substrate may be at least partially honeycombed in shape. The catalytic surface may be disposed on at least a portion of the substrate. The catalytic surface may be coated on, bonded to, impregnated on, coupled to and/or a portion of the substrate. The doped titanium catalyst of the catalytic surface may include approximately 5% rhodium, approximately 0.5 mol % copper, and/or approximately 5 mol % antimony. The doped titanium catalyst comprises a ratio of rhodium to antimony of approximately 1:approximately 1, in some implementations. The doped titanium catalyst may include approximately equal parts by mol of silver, rhodium, antimony, and less copper by mol than silver. In some implementations, the disinfecting system may be incorporated into one or more ducts of an air management system. For example, the disinfecting system may be included in an air conditioning and/or heating system, included in a standalone air purifier, or incorporated in a backpack disinfecting system. In various implementations, the oxygen level of the disinfected air may be increased (e.g., when compared to the air as it enters the channels of the substrate). The amount of hydrogen peroxide produce may include at least approximately 5 ppb by volume. The amount of hydrogen peroxide produce comprises approximately 10 ppb by volume to approximately 30 ppb by volume. The amount of excess ozone produced during generation of the hydrogen peroxide may be low. The amount of excess ozone produced during generation of the hydrogen peroxide may be undetectable by smell. The produced hydrogen peroxide may be carried by the air away from the substrate and may disinfect surfaces that contact the hydrogen peroxide in the air exiting the substrate (e.g., channels of the substrate).

In various implementations, air and/or surfaces may be disinfected using the described systems. UV lamp(s) may emit at least a first band of UV light and a second band of UV light. The first band of UV light comprises approximately 253.7 nm and the second band of UV light comprises approximately 185 nm. The light emitting by the UV lamp(s) may be at least partially emitted to the sleeve(s). The sleeve(s) may include a first zone and a second zone. The first zone may allow the first band of UV light to pass through the first zone and inhibit the second band of UV light from passing through the first zone. The second zone allows the second band of UV light and the first band of UV light to pass through the second zone. In some implementations, the first zone and/or the second zone may inhibit other bands of light (e.g., by absorbing and/or not allowing bands of light other than 253.7 nm and/or the second band of UV light comprises approximately 185 nm to pass). At least a portion of the light emitted may be allowed to pass through at least one of the first zone or the second zone. The light passing through the sleeve(s) may shine on at least a portion of a substrate. The substrate may include channels (e.g., that extend through the substrate). A catalytic surface may be disposed on at least a portion of the substrate. The catalytic surface may include a doped titanium dioxide catalyst. The doped titanium dioxide catalyst may include titanium doped with approximately 1 mol % to approximately 25 mol % silver, approximately 1 mol % to approximately 25 mol % rhodium, approximately 0.1 mol % to approximately 2 mol % copper, and approximately 1 mol % to approximately 25 mol % antimony. Air may pass through one or more of the channels of the substrate (e.g., to be disinfected and/or to carry generated hydrogen peroxide to other surfaces to disinfect). Hydrogen peroxide may be generated by contacting the air with the catalytic surface and the UV light proximate the substrate.

Implementations may include one or more of the following features. In various implementations, the oxygen level of the disinfected air may be increased (e.g., when compared to the air as it enters the channels of the substrate). The amount of hydrogen peroxide produce may include at least approximately 5 ppb by volume. The amount of hydrogen peroxide produce comprises approximately 10 ppb by volume to approximately 30 ppb by volume. The amount of excess ozone produced during generation of the hydrogen peroxide may be low. The amount of excess ozone produced during generation of the hydrogen peroxide may be undetectable by smell. The produced hydrogen peroxide may be carried by the air away from the substrate and may disinfect surfaces that contact the hydrogen peroxide in the air exiting the substrate (e.g., channels of the substrate).

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates an implementation of an example reactor unit.

FIG. 2 illustrates an implementation of an example spliced UV lamp.

Like reference symbols in the various drawings indicate like elements.

### DETAILED DESCRIPTION

In various implementations, systems and processes may disinfect air and/or surface(s). Oxidizing molecules, from the described catalytic synthesis, may be dispensed into ambient air of occupied workplaces, homes, or rooms with public attendance, for example. The dispersal may disinfect the air, surfaces, and/or portions thereof.

In various implementations, the disinfecting system may include catalyst and a UV lamp that in the presence of air (e.g., air that has a humidity of at least approximately 10 percent relative humidity produces hydrogen peroxide. The amount of hydrogen peroxide produced may be in quantities sufficient (e.g., at least approximately 5 ppb hydrogen peroxide by volume) to disinfect air and/or surfaces of predetermined pathogens (e.g., viruses such as coronaviruses and/or influenza; bacteria such as E. coli and/or salmonella; fungi; and/or other pathogens) and/or predetermined volatile organic compounds (VOC).

In various implementations, the hydrogen peroxide may be produced without excess ozone. Since ozone is a known irritant, the reduction of ozone during disinfection may increase user satisfaction and/or compliance with government, industry, and/or facility standards (e.g., US OSHA acceptable limit for ozone in occupied spaces). Currently known methods of hydrogen peroxide production tend to produce a suboptimal rate of ozone, which is considered a pollutant, to the effective and more environmentally friendly (to the atmosphere and people) oxidizer hydrogen peroxide. For example, currently known methods release ozone and fail to generate enough hydrogen peroxide to disinfect surfaces and/or the air.

In various implementations, the system and processes may utilize water (e.g., gaseous water) H₂O and molecular oxygen O₂ present in ambient air (e.g., gaseous water) to produce hydrogen peroxide in a photocatalytic process. Photocatalysis or photogenerated catalysis accelerates a photoreaction in the presence of a catalyst. Photons from a light source are absorbed on the surface of a catalytic substrate, creating electron-hole pairs, (e.g., an exciton, h⁺+e⁻). In the described systems and processes, a metal oxide, titanium dioxide (TiO₂ or titanium (IV) oxide) and/or doped titanium dioxide is involved as catalyst in the described reactions as follows:

TiO₂+hv→TiO₂+h⁺+e⁻ (A)

where hv represents the energy of the photon, Eₚₕₒₜₒₙ,
h is the Planck constant, and
v is the frequency of light, which is inverse proportional to the wavelength, λ(v=c/λ, where c is the constant of speed of light in vacuum).

Photocatalytic reactions between generated holes and reductants, produce oxidized products, as follows:

h⁺+H₂O→H⁺+.OH

2h⁺+2H₂O→2H⁺+H₂O₂

H₂O₂→2.OH (B)

while reactions between exited electrons and oxidants produce reduced products, as follows:

e⁻+O₂→.O₂⁻

.O₂⁻+H₂O+H⁺→H₂O₂+O₂

H₂O₂→2.OH (C)

and combinations thereof, participating in multiple chemical transformations (e.g., first set of reactions (A), the second set of reactions (B), the third set of reactions (C), and/or other reactions). Such multistep reaction mechanisms, and/or catalytic cycles may include the said and other hydrogen/oxygen species (e.g., .OH, .O₂⁻, O₃⁻, HO₂⁻) (e.g., in electrically neutral and/or in ion states; and/or with or without unpaired valence electrons; presenting as highly reactive radicals or not), to thereby define a “TiO₂/UV” reaction (e.g., Reactions B, Reactions C, Reactions A-B, or Reactions A and C), and said hydrogen/oxygen species to define a plurality of “TiO₂/UV” hydrogen/oxygen species (e.g., Reactions A-C). Often, although intermediates are detected, the specific mechanisms about how the actual elementary reactions occur are unknown.

The emitted light in the described photocatalytic reactions may include ultraviolet (UV) light/radiation. The electromagnetic spectrum of ultraviolet (UV) radiation ranges from long-wavelength of 400 nanometers to short-wavelength (UVC) of 10 nanometers and a range of photon energy of 3.1 to 124 eV across the UV spectrum. The UV lamp selected for use in the described process may be selected such that predetermined wavelength(s) and/or ratio(s) of wavelengths are allowed to pass. UVC light of approximately 185 nanometers may create ozone from molecular oxygen (e.g., in ambient air) by means of photolysis or photodecomposition (e.g., chemical reaction in which a chemical compound is broken down by photons), as follows:

O₂+hv_{(<242 nm)}→2.O (D)

where each oxygen atom radical, .O then quickly combines with a molecular oxygen molecule to form ozone as follows:

.O+O₂→O₃ (E)

These reactions (Reactions D-E) may define an “UV/O₃” reaction.

The presence of ozone, O₃ in the TiO₂/UV reaction cycle may increase the yield rate of the photocatalytic process when compared with a TiO₂/UV reaction cycle without the presence of ozone. The presence of ozone may enable, for example, the following:

e⁻+O₃→.O⁻+O₂

e⁻+O₂→.O₂⁻

.O₂⁻+O₃→O₂+.O₃⁻ (F)

as intermediate pathways. These species (in Reactions F), together with the TiO₂/UV reaction (Reactions C) or TiO₂/UV hydrogen/oxygen species (hydrogen/oxygen species of Reactions C) may provide an enhanced electron capture rate by reducing the recombination rate of photoexcited electron-hole pairs and, as a result, may increases the photocatalytic yield rate, to thereby define a “TiO₂/UV/O₃” reaction (Reactions C-F), said hydrogen/oxygen species together to define a plurality of “TiO₂/UV/O₃” hydrogen/oxygen species (hydrogen/oxygen species in Reactions C-F).

In various implementations, the disinfecting systems and processes may utilize operations of Reactions A-F, or portions thereof, to generate hydrogen peroxide at rates that allow disinfection of air and/or surfaces. Reactions A-B may also occur to some extent along with Reactions A, C-F, in some implementations, with to the availability of the excited holes. In some implementations, the titanium catalyst utilized by the disinfecting system and processes may include doped titanium dioxide. The doped titanium dioxide catalyst may increase (e.g., when compared to non-doped titanium dioxide catalysts) the yield of hydrogen peroxide generated and/or, in implementations utilizing the combined Reactions C-F, reduce the amount of excess ozone (e.g., amount of ozone produced that is greater than the amount used in the production of hydrogen peroxide).

In various implementations, the titanium dioxide of the catalyst may be doped with Silver, Rhodium, Copper, and/or Antimony. Silver in doped titanium dioxide may increase the yields of hydrogen peroxide (e.g., when compared with non-doped titanium dioxide), due to its high oxidation power and/or high oxidation potentials. Silver in doped titanium dioxide may increase the electronegativity of other catalysts, preserve the anatase form of titanium dioxide in higher temperatures, may absorb UV and visible light, and/or increase photooxidation. Rhodium in doped titanium dioxide may increase catalytic dehydrogenative cross-coupling (e.g., when compared with non-doped titanium dioxide); and/or attracts and/or attaches to water (e.g., due to its hydrophilic properties) to increase and/or promote generation of a liquid water film on the catalytic surface. In some implementations, the catalytic surfaces and/or substrates are doped with mesoporous Rhodium nanoparticles, which may allow catalytic reactions inside the hollow pores of the Rhodium. Copper, the in doped titanium dioxide catalyst, depending on its oxidation state may efficiently catalyze reactions involving one and two-electron (radical and polar) mechanisms (e.g., when compared with non-doped titanium dioxide); and/or increase exciton lifetimes which increases the photocatalytic activity. Antimony in the doped titanium dioxide catalyst (e.g., when compared with non-doped titanium dioxide), may be an multielectron transfer sensitizer for photocatalysis and artificial photosynthesis, which may increase photocatalytic activity enhancing visible region absorption. This may induce lone pair surface electronic states which may trap the holes at the lone pair surface sites; and thus, inhibit the recombination of electrons and holes generated in an initial photoexcitation step to increase the yield of hydrogen peroxide generation using the system. The doping of a titanium dioxide catalyst with one of the following: Silver, Rhodium, Copper, and Antimony may increase the yield of hydrogen peroxide generated and/or, reduce the amount of excess ozone, while doping with any combination of such substances or with all of such substances may further increase the yield of hydrogen peroxide generated and/or, further reduce the amount of excess ozone.

The titanium dioxide may be doped with approximately 1 mol % to approximately 25 mol % Silver. In some implementations, the titanium dioxide may be doped with approximately 5 mol % Silver. The titanium dioxide may be doped with approximately 1 mol % to approximately 25 mol % Rhodium. In some implementations, the titanium dioxide may be doped with approximately 5 mol % Rhodium. The titanium dioxide may be doped with approximately 0.1 mol % to approximately 2 mol % Copper. In some implementations, the titanium dioxide may be doped with approximately 0.5 mol % Copper. The titanium dioxide may be doped with approximately 1 mol % to approximately 25 mol % Antimony. In some implementations, the titanium dioxide may be doped with approximately 5 mol % Antimony.

In some implementations, the catalyst may include titanium dioxide doped with Silver, Copper, Rhodium, and Antimony. The use of the four-element doped titanium dioxide may provide unexpected results when compared with the results from three element or single doped titanium dioxide. For example, this described four element doped titanium dioxide may produce approximately 15% to approximately 40% more hydrogen peroxide than titanium dioxide alone as a catalyst. In some implementations, the ratio of Rhodium to Antimony may be approximately 1:approximately 1. In some implementations, the titanium dioxide may be doped with approximately equal parts (by mol) of Silver, Rhodium, and Antimony and less Copper (by mol) than Silver. The amount of Copper (by mol) may be less than the amount of Silver, Rhodium, and/or Antimony, individually, in the doped titanium dioxide. In some implementations, the doped titanium dioxide may include approximately 5 mol % Silver, approximately 5 mol % of Rhodium, approximately 0.5 mol % of Copper, and approximately 5 mol % of Antimony.

The doped titanium dioxide may be coated on, impregnated on, and/or otherwise coupled to or a portion of a substrate of the disinfection system. The doped titanium dioxide may form at least a portion of the substrate, in some implementations. The substrate may include any appropriate material such as a metal(s) like Aluminum; plastic(s), and/or composite(s). The substrate may not substantially deteriorate in the presence of UV light. The substrate may have a shape and/or geometry to allow a predetermined amount of substrate exposure (e.g., to UV light). In some implementations, the geometry of the substrate may have a surface area greater than a planar substrate of the same dimensions (e.g., length, width, and/or height). For example, the geometry chosen may have at least 30% greater surface area than a similarly dimensions flat surface. Reducing the dimensions of the substrate may reduce costs (e.g., since fewer UV lamps may be utilized) and/or impairments to portability. The substrate may have openings (e.g., holes) and/or channels (e.g., tracks, recesses, etc.) to allow air to control air flow relative to the substrate (e.g., flow through and/or over the substrate). For example, at least a portion of the substrate may have a grid pattern, such as a honeycomb, rectangular, and/or circular grid pattern. As another nonlimiting example, at least a portion of the substrate may be pleated. As another nonlimiting example, at least a portion of the substrate may include fins.

The system may also include Ultraviolet (UV) lamp(s). The UV lamp may include a low pressure mercury vapor lamp. In some implementations, the UV lamp may include light emitting diode (LED) lamps. The substrate may be exposed to the Ultraviolet (UV) light generated by the UV lamp(s). The shape of the substrate (e.g., grid patterned, such as honeycombed) may allow a greater surface area to be exposed to the UV lamp(s) than if a planar, similarly dimensioned substrate was utilized.

A UV lamp may include an outer sleeve, a chamber inside the sleeve that houses UV lamp components (e.g., filament, mercury vapor, etc.), and ballast(s). The outer sleeve may include quartz and fused silica. The outer sleeve may be a unibody or multi-component.

The electromagnetic spectrum of ultraviolet (UV) radiation ranges from long-wavelength of 400 nanometers to short-wavelength (UVC) of 10 nanometers and a range of photon energy of 3.1 to 124 eV across the UV spectrum. The described systems and processes may utilize, low-pressure and/or medium-pressure mercury vapor lamps, in which UV light may be emitted predominantly in two UVC bands at approximately 253.7 nanometers, nm and at approximately 185 nanometers, nm are emitted. When the UV lamp includes sleeves that include quartz, the quartz sleeve portion allows the longer wavelength UVC radiation (e.g., approximately 253.7 nm) to pass and blocks the shorter wavelength UVC radiation (e.g., approximately 185 nm) by absorbing the shorter wavelength. Sleeve(s) or portions thereof that include fused silica glass may not substantially absorb either of these UVC bands and therefore, may allow the shorter wavelength UVC radiation (e.g., approximately 185 nm) and the longer wavelength UVC (e.g., approximately 253.7 nm) to pass through the sleeve.

The sleeve of the UV lamp may be a dual zoned sleeve, in various implementations. As a nonlimiting example, the sleeve may include a quartz sleeve and a fused silica sleeve fused and/or spliced together. The splice between the quartz portion of the sleeve and the fused silica portion of the sleeve may be a fusing zone. The full length of the fused silica portion may be exposed when fused sleeves are utilized to create a dual zoned UV lamp.

In some implementations, the dual zoned sleeve may include a first sleeve partially disposed in a second sleeve. The outer sleeve of the UV lamp may include a first member that includes fused silica sleeve and a second member that includes a quartz sleeve. The diameter of the quartz sleeve may be greater than the fused silica sleeve such that the fused silica sleeve may be disposed partially in the quartz portion. The quartz sleeve may be disposed about the fused silica sleeve (e.g., the fused silica sleeve may be disposed at least partially in the quartz sleeve). The length of the fused silica sleeve exposed (e.g., not disposed in the be second quartz sleeve) may be less than the length of the quartz sleeve. For example, while the overall length of the fused silica sleeve may be less than, equal to, and/or greater than the quartz sleeve, the length of the fused silica sleeve exposed (e.g., not disposed in the quartz sleeve) may be less than the length of the quartz sleeve.

The dual zoned UV lamp may produce two bands of Ultraviolet-C light (UVC or short wave UV light). The UV lamp may produce a first band at approximately 185 nm and a second band at 253.7 nm. The production of the two band UVC light may be allowed by use of dual zoned sleeve that includes quartz portion and fused silica portion. The ratio of quartz to fused silica in the sleeve and/or the positioning of a splice line between sections may allow generation of the two band UVC and control the ratio of 185 nm light to 253.7 nm light emitted by the UV lamp.

The quartz portion of the sleeve may at least partially cover the mercury arc length in a mercury lamp. In some implementations, the quartz portion of the outer sleeve may cover the mercury arc length. The sleeve may include approximately 1% by length to approximately 5% by length of exposed fused silica relative to the overall length of the sleeve (e.g., combined quartz and fused silica sleeve). The sleeve may include approximately 2 to approximately 3% exposed used silica by length (e.g., approximately 2.5%) and approximately 97% to approximately 98% (e.g., 97.5%) quartz by length (e.g., in a sleeve of uniform diameter, the sleeve may have a cross section with a diameter and a length normal to the cross-section). The quartz sleeve may absorb the ozone generating shorter wavelength UVC radiation band at approximately 185 nm that is emitted from the UV lamp and/or passes through the portion of the silica sleeve disposed in the quartz sleeve. In some implementations, use of approximately 97.5% by length of quartz relative to the overall length of the sleeve may generate approximately 155 ml of excess ozone that reverts molecular oxygen and boosts the oxygen in the air exiting the system.

In some implementations a two-stage reaction process may be utilized, which may increase the yield of hydrogen peroxide and reduce excess ozone, stage one to include a photolytic reaction, and stage two to include a photocatalytic reaction. In some implementations, the emitted UVC light may allow a photolytic reaction involving UV and O₃ and this photolytic reaction may allow the photocatalytic reaction involving TiO₂ and UV to be supplemented or replaced with a reaction involving TiO₂, UV, and O₃, which may increase the yield of hydrogen peroxide and reduce excess ozone. In some implementations, using a two zoned mercury vapor UV lamp may induce the photolysis of molecular oxygen (e.g., to produce ozone from ambient air) and the photocatalytic reaction or reaction cycle to produce TiO₂/UV/O₃ oxygen/hydrogen species required to synthesize hydrogen peroxide, H₂O₂. The amount of (a) radiation photon energy produced by oxygen producing wavelength of ultraviolet light in the UVC band at approximately 185 nanometers, nm to (b) the radiation photon energy produced by non-oxygen producing wavelength of ultraviolet light in the UVC band at approximately 253.7 nanometers, nm, is controlled by the ratio of the length of quartz tube portion to the length of exposed to the length of the fused silica glass tube portion of the sleeve of the UV lamp (e.g., a ratio of the light energy of the first UVC band to the light energy of the second UVC band correlates to a stoichiometric quantification ratio of reactants, in particular an amount of ozone to other oxygen, hydrogen and hydrogen/oxygen species). The control of the stoichiometric quantification ratio of the reactions may increase the yield rate of generated hydrogen peroxide while decreasing the environmental disbursement of ozone due to excess ozone. The excess ozone may be reduced to an amount below an acceptable limit for occupied spaces, in some implementations, by control of the ratio of the light energy of the first UVC band to the light energy of the second UVC band.

In some implementations, the catalytic yield rate of the reactor units producing hydrogen peroxide, H₂O₂ is increased by using the described spliced sleeve with a mercury arc UV lamp. Since the described systems and processes include the utilization of approximately 185 nanometers wavelength UVC light to create ozone from diatomic molecular oxygen in ambient air (e.g., by means of photolysis or photodecomposition), the ratio between a quartz tube portion and a silica glass tube portion quartz defines a ozone production rate, which correlates to the stoichiometric quantification of reactants that produce the increased a catalytic yield rate of the described systems and processes.

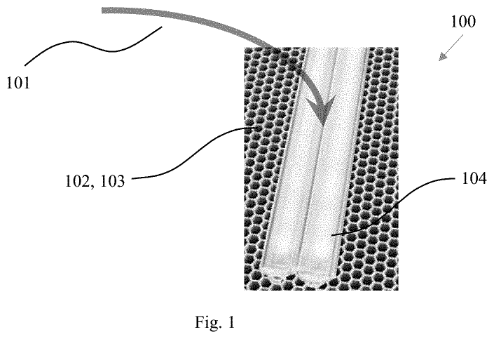

The described systems and processes may be implemented in any appropriate manner to allow hydrogen peroxide generation for disinfection (e.g. of surfaces and/or air). For example, a disinfecting unit may be placed in duct(s) and/or outlets of an air management system or a heating, ventilation, and air conditioning (HVAC) system. As an example, FIG. 1 illustrates an implementation of an example a disinfection unit 100. Ambient air 101 is directed to flow through a catalytic converter 102 (e.g., a substrate and catalyst). The ambient air 101 may or may not be forced (e.g., by use of a fan) through the disinfecting unit. As illustrated the catalytic converter 102 may be honeycomb-shaped and include channels through which the ambient air may flow. The catalytic surface 103 of the catalytic converter 102 may be exposed to UV light generated by a UV lamp 104. Thus, as the ambient air passes through the catalytic converter, the ambient air may participate in Reactions A-B and/or catalytic Reactions A, C-F to generate hydrogen peroxide. The resulting air and generated hydrogen peroxide may flow out of the disinfecting unit to allow disinfection of surfaces and/or air. The generated hydrogen peroxide may be at least 5 parts per billion (ppb) by volume. The generated hydrogen peroxide in the resulting air (e.g., air exiting the disinfecting unit) may be at approximately 10 ppb by volume to approximately 30 ppb by volume. The resulting air may have a low enough level of excess ozone that it is undetectable by smell and/or such that it complies with regulations (e.g., governmental, industry, etc.).

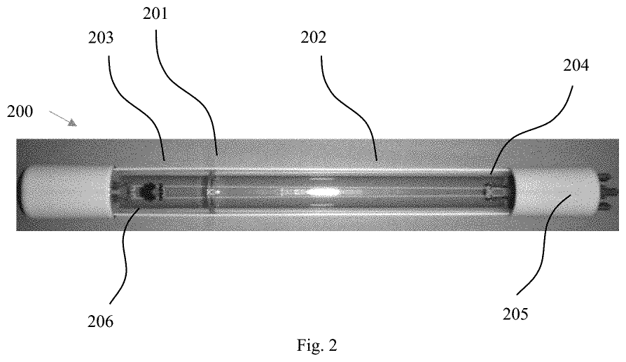

FIG. 2 illustrates an implementation of a UV lamp that can be used with the disinfecting unit 100. The UV lamp may include a mercury arc UV lamp 200. The UV lamp 200 includes a splice 201 represents a fusing zone between a quartz tube portion 202 and a fused silica glass tube portion 203 of the sleeve. As illustrated, the ratio by length is approximately 20/80 of fused silica tube to quartz. The UV lamp 200 includes an electrode 204 and an electrical connector socket 205. When the lamp is not electrically powered, a mercury cold spot 206 may be visible.

In the UV lamp 104, 200, the quartz tube portion 202 of a spliced sleeve may absorb at least a portion of the ozone generating wavelength UVC radiation band (e.g., approximately 185 nm). In some implementations, a quartz glass sheath tube (not shown) may at least partially surround at least a portion of a fused silica sleeve of the UV lamp tube to absorb ozone generating wavelength UVC radiation band (e.g., approximately 185 nm).

In various implementations, a system and method for disinfection may decomposing microbial pathogens and/or volatile organic compounds. The process may include a heterogeneous photocatalytic reaction cycle synthesizing gaseous hydrogen peroxide from air utilizing a catalytic surface of the described doped titanium dioxide catalyst. The air includes gaseous water molecules and molecular oxygen. The doped titanium dioxide catalyst may increase a yield rate of the heterogeneous photocatalytic reaction cycle and synthesize hydrogen peroxide. The doped titanium dioxide catalyst may include one or more transition metal and/or metalloid substances as additives and may provide one or more of the following features one and two-electron radical and polar mechanisms, enhanced exciton lifetimes, catalytic activation of the electronegativity of catalytic substances, enhanced catalytic dehydrogenative cross-coupling to thereby enhance hydrophilic properties attracting and attaching water and increasing a liquid film on the catalytic surface, and/or inducement of lone pair surface electronic states trapping the holes at the lone pair surface sites thus inhibiting the recombination of electrons and electron holes generated in an initial photoexcitation step.

Implementations may include one or more of the following features. The metalloid substance may be Antimony. The effect of enhanced catalytic dehydrogenative cross-coupling to thereby enhance hydrophilic properties attracting and attaching water and increasing a liquid film on the catalytic surface may be provided by titanium oxide doped with at least Rhodium. The Rhodium of the doped titanium dioxide catalyst may include mesoporous nanoparticles. The one or more transition metal and/or metalloid substances as additives may include approximately 0.5 mol % of Copper, approximately 5 mol % of Silver, approximately 5 mol % of Rhodium; and about 5 mol % of Antimony. The balance may be titanium dioxide in some implementations (e.g., approximately 84.5%). In some implementations, the disinfecting system and processes may include a photolytic reaction of a plurality of molecular oxygen to produce ozone from ambient air. The generated hydrogen peroxide may be dispensed to an environment for decomposing microbial pathogens and/or volatile organic compounds. In some implementations, the described systems and methods may allow environmental air to participate in a photolytic reaction and, thereafter, a heterogeneous photocatalytic reaction cycle stage, where a catalytic substance is exposed to UV light, to increase the yield rate of produced gaseous hydrogen peroxide. The gaseous hydrogen peroxide may be dispensed in an environment to decompose microbial pathogens and/or volatile organic compounds. In some implementations, a disinfecting unit of the system may include at least one UV lamp. The use of the UV lamp may increase the yield of generated hydrogen peroxide since the UV lamp may allow a photolytic reaction of molecular oxygen to produce ozone from ambient air and a heterogeneous photocatalytic reaction cycle that synthesizes hydrogen peroxide molecules from water, molecular oxygen, and ozone. The UV lamp may emit UV radiation at a UVC band of about at approximately 185 nm to induce the photolytic reaction, and a second UV radiation at approximately 253.7 nanometers. The UV lamp may be a spliced dual zone mercury vapor UV lamp, comprising a quartz tube portion and a fused silica glass tube portion. The quartz tube and the fused silica glass tube may be spliced into a sleeve and/or the quartz tube may cover at least a portion of the fused silica tube. The mercury arc length of the UV lamp may be covered by the quartz portion of the tube and not the fused silica tube, in some implementations. The ratio between the quartz tube portion and the fused silica glass tube portion may control the ratio of the emission energy of the first UV radiation and the emission energy of the second UV radiation. The length of the quartz tube portion may be approximately 97.5% of the overall tube length and the length of the exposed portion of the fused silica glass portion may be approximately 2.5% of the overall tube length. The ratio of the quartz tube portion and the fused silica glass portion may control the first yield rate of the photolytic reaction that increases the second yield rate of the heterogeneous photocatalytic reaction cycle.

The ambient air may have a relative humidity of approximately 20 percent relative humidity to 100 percent relative humidity. The ambient air may be exposed to UV light from the UV lamp that is emitted onto the substrate and may undergo the described photocatalytic reactions (e.g., Reactions A-B and/or Reactions A, C-F) and/or other reactions.

In various implementations, the systems and processes may be incorporated into the ductwork of air management systems; heating, ventilation, and air conditioning (HVAC) systems; and/or in a stand-alone configuration that includes a fan, into the circulation of ambient air. The systems and processes may be incorporated into a movable unit, such as a cart, a backpack, and/or handheld unit.

In some implementations, the use of the UV light in the system in the production of hydrogen peroxide rather than in direct disinfectant of air and/or surfaces may increase safety of the space. For example, surfaces may not be directly exposed to the UV light which may decrease wear and/or people may not be directly exposed to the UV light which may increase user and occupant safety.

In various implementations, the described systems and processes may be capable of producing continuous, semi-continuous and/or manually controlled photocatalytic synthetization of gaseous hydrogen peroxide, from humidity (e.g., gaseous water and diatomic molecular oxygen) available in ambient air, for air and/or surface disinfection.

Currently, there has not been recognition of methods and systems in the field of continuous air and surface pathogen reduction in occupied spaces. Often processes claimed to have disinfection capabilities fail to produce hydrogen peroxide in quantities that are capable of disinfecting, such as producing at least 5 ppb of hydrogen peroxide in a resulting air stream from the disinfecting unit. The disclosed systems and methods provide, a previously unknown, alternative pathway of synthesizing hydrogen peroxide on described catalytic surfaces by intentionally generating and facilitating ozone (a by-product in an ultraviolet light driven photocatalytic method) to thereby increase the hydrogen peroxide synthetization yield rate of the overall catalytic reaction, while keeping the ozone dispense rate of the disinfecting system below an acceptable limit for continuous use in occupied space.

Catalysis means, for example, a process of increasing the rate of the chemical transformation of one set of chemical substances to another and/or favoring a particular reaction and/or chirality, by adding a substance known as a catalyst. In the transformation the substances may encompass changes that only involve the positions of electrons in the forming and breaking of chemical bonds between atoms, with no change to the nuclei. The catalytic surface may facilitate the transformation, i.e. the catalyzed reaction repeatedly, while the catalytic substance is usually not being consumed. The catalyst may provide an alternative reaction pathway with a lower activation energy than the non-catalyzed reaction route, forming a temporary intermediate, which then regenerates the original catalyst in a cyclic process. In a heterogeneous catalytic process, the molecules of the catalyst may not be in the same phase as the reactant substances, as gases or liquids are adsorbed onto the surface of a solid phase catalytic substance. Photocatalysis or photogenerated catalysis means, for example, the acceleration of a photoreaction in the presence of a catalyst,

The system(s) and method(s) provided by the various embodiments of present invention comprise several independent inventive features providing substantial improvements. The greatest benefit will be achieved in the field of continuous air and surface pathogen reduction in occupied spaces, and more particularly in the field of continuous photocatalytic synthetization of gaseous hydrogen peroxide, from humidity (e.g., gaseous water in air) and molecular oxygen (e.g., from ambient air) for air and surface disinfection.

In various implementations, other operations may be used with the described systems and processes to synthesize and/or dispose safe low level traces of hydrogen peroxide, H₂O₂ for microbial control in occupied spaces as described in U.S. patent application Ser. No. 12/187,755 (published as U.S. Pub. No. 2009/0041617), which is incorporated herein by reference to the extent that it does not conflict with the teachings herein. However, while U.S. patent application Ser. No. 12/187,755 describes gaseous hydrogen peroxide, H₂O₂ that is substantially free of, for example, hydration, ozone, plasma species, and/or organic species; it is silent in teaching what taught embodiments, or combinations thereof, one skilled in the art could practically achieve that goal, without reducing substantially the yield rate of the catalytic process. The embodiments disclosed in U.S. patent application Ser. No. 12/187,755 fail to teach production of an effective amount of hydrogen peroxide while substantially free of ozone.

Although the substrate is described including at least a portion that is honeycombed, the substrate may have any appropriate shape. For example, the substrate may include a pleated portion with channels and/or holes for air passage. The substrate may include circular

Hydrogen peroxide, as described, is also commonly known as H₂O₂ dihydrogen dioxide. Water, as described, is commonly known as H₂O or dihydrogen oxide. Diatomic molecular oxygen, as described, is commonly known as O₂ or dioxide. Ozone, as described, is also commonly known as O₃ or trioxygen

In various implementations, described process(es) may be implemented by various described system(s), such as system 100. In addition, various described operation(s) may be added, deleted, and/or modified in implementations of the described process(es) and/or system(s). In some implementations, a described process or operations thereof may be performed in combination with other described process(es) or operations thereof.

It is to be understood the implementations are not limited to particular systems or processes described which may, of course, vary. It is also to be understood that the terminology used herein is for the purpose of describing particular implementations only, and is not intended to be limiting. As used in this specification, the singular forms “a”, “an” and “the” include plural referents unless the content clearly indicates otherwise. Thus, for example, reference to “a tubing” includes a combination of two or more tubing and reference to “a quartz lamp” includes different types and/or combinations of quartz lamps.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A disinfecting system comprising:
a mercury arc lamp;
a dual-zoned sleeve comprising:
a first zone comprising quartz;
a second zone comprising fused silica, wherein at the second zone extends along approximately 1% to approximately 5% of a length of the dual-zoned sleeve;
wherein the mercury arc lamp is disposed at least partially in a lumen of the sleeve;
a substrate comprising:
one or more channels;
a catalytic surface on at least a portion of the substrate, wherein the catalytic surface comprises a doped titanium dioxide catalyst, wherein the doped titanium dioxide catalyst comprises:
approximately 1 mol % to approximately 25 mol % silver;
approximately 1 mol % to approximately 25 mol % rhodium;
approximately 0.1 mol % to approximately 2 mol % copper; and
approximately 1 mol % to approximately 25 mol % antimony;
wherein at least a portion of the substrate is exposed to light passing through the sleeve;
wherein air that passes though one or more of the openings in the substrate is at least partially disinfected by hydrogen peroxide generated by exposure of the air to the catalytic surface and the light passing through the sleeve.

2. The disinfecting system of claim 1 wherein the mercury lamp comprises a low-pressure mercury vapor lamp.

3. The disinfecting system of claim 1 wherein a length of the second zone that is exposed comprises approximately 2.5% of a length of the dual-zoned sleeve.

4. The disinfecting system of claim 1 wherein the doped titanium catalyst comprises approximately 5% rhodium, approximately 0.5 mol % copper, and approximately 5 mol % antimony.

5. The disinfecting system of claim 1 wherein the doped titanium catalyst comprises a ratio of rhodium to antimony of approximately 1:approximately 1.

6. The disinfecting system of claim 1 wherein the doped titanium catalyst comprises approximately equal parts by mol of silver, rhodium, antimony, and less copper by mol than silver.

7. The disinfecting system of claim 1 wherein the catalytic surface comprises the doped titanium catalyst coated on, impregnated on, or coupled to at least a portion of one or more surfaces of the substrate.

8. The disinfecting system of claim 1 wherein the channels of the substrate form a honeycomb.

9. A disinfecting system comprising:
one or more UV lamps, wherein each UV lamp is capable of emitting at least a first band of UV light and a second band of UV light, wherein the first band of UV light comprises approximately 253.7 nm, and wherein the second band of UV light comprises approximately 185 nm.
one or more sleeves comprising:
a first zone, wherein the first zone allows the first band of UV light to pass through the first zone and inhibits the second band of UV light from passing through the first zone;
a second zone comprising, wherein the second zone allows the second band of UV light and the first band of UV light to pass through the second zone;
wherein at least a portion of the light is emitted to at least a portion of the one or more sleeves such that at least a portion of the light emitted is allowed to pass through at least one of the first zone or the second zone;
a substrate comprising:
one or more channels;
a catalytic surface on at least a portion of the substrate, wherein the catalytic surface comprises a doped titanium dioxide catalyst, wherein the doped titanium dioxide catalyst comprises:
approximately 1 mol % to approximately 25 mol % silver;
approximately 1 mol % to approximately 25 mol % rhodium;
approximately 0.1 mol % to approximately 2 mol % copper; and
approximately 1 mol % to approximately 25 mol % antimony;
wherein at least a portion of the substrate is exposed to light passing through the one or more sleeves;
wherein air that passes though one or more of the channels in the one or more substrates is at least partially disinfected by hydrogen peroxide generated by exposure of the air to the catalytic surface of the one or more substrates and the light passing through the one or more sleeves.

10. The disinfecting system of claim 1 wherein at least one of the sleeves comprises a dual-zoned sleeve comprising the first zone and the second zone spliced together.

11. The disinfecting system of claim 1 wherein the second zone has a length of approximately 1% to approximately 5% of the length of the dual-zoned sleeve.

12. The disinfecting system of claim 1 wherein the one or more sleeves comprise an outer sleeve and an inner sleeve, and wherein the outer sleeve comprises the first zone and the inner sleeve comprises the second zone, and wherein the inner sleeve is disposed partially in the outer sleeve.

13. The disinfecting system of claim 11 wherein length of the inner sleeve that is not disposed in the outer sleeve is less than the length of the outer sleeve.

14. The disinfecting system of claim 11 wherein the disinfecting system is incorporated into one or more ducts of an air management system.

15. A method of disinfecting air, the method comprising:
allowing one or more UV lamps to emit at least a first band of UV light and a second band of UV light, wherein the first band of UV light comprises approximately 253.7 nm, and wherein the second band of UV light comprises approximately 185 nm;
wherein light emitting by the one or more UV lamps is at least partially emitted to one or more sleeves, wherein the one or more sleeves comprise:
a first zone, wherein the first zone allows the first band of UV light to pass through the first zone and inhibits the second band of UV light from passing through the first zone; and
a second zone comprising, wherein the second zone allows the second band of UV light and the first band of UV light to pass through the second zone;
wherein at least a portion of the light emitted from the one or more UV lamps is allowed to pass through at least one of the first zone or the second zone;
allowing light passing through the one or more sleeves to shine on at least a portion of a substrate, wherein the substrate comprises:
one or more channels; and
a catalytic surface on at least a portion of the substrate, wherein the catalytic surface comprises a doped titanium dioxide catalyst, wherein the doped titanium dioxide catalyst comprises:
approximately 1 mol % to approximately 25 mol % silver;
approximately 1 mol % to approximately 25 mol % rhodium;
approximately 0.1 mol % to approximately 2 mol % copper; and
approximately 1 mol % to approximately 25 mol % antimony;
allowing air to pass through one or more of the channels of the substrate, and wherein contact with the catalytic surface and the UV light proximate the substrate generates hydrogen peroxide to at least partially disinfects the air.

16. The method of claim 15 wherein the oxygen level of the disinfected air is increased.

17. The method of claim 15 wherein the amount of hydrogen peroxide produce comprises at least approximately 5 ppb by volume.

18. The method of claim 15 wherein the amount of hydrogen peroxide produce comprises approximately 10 ppb by volume to approximately 30 ppb by volume.

19. The method of claim 15 wherein the amount of excess ozone produced is undetectable by smell.

20. The method of claim 15 wherein the produced hydrogen peroxide is carried by the air away from the substrate and disinfects surfaces that contact the hydrogen peroxide in the air exiting the substrate.
